(19) United States
(12) Patent Application Publication
(10) Pub. No.: US 2016/0299160 A1
(43) Pub. Date: Oct. 13, 2016

(54) PORTABLE CHEMICAL TESTING APPARATUS, SYSTEM, AND METHOD

(71) Applicant: MWD TECHNOLOGIES, INC., Round Rock, TX (US)

(72) Inventors: Christian C. DiRuggiero, Round Rock, TX (US); Crispino DiRuggiero, Round Rock, TX (US); Michelle W. DiRuggiero, Round Rock, TX (US)

(73) Assignee: MWD TECHNOLOGIES, INC., Round Rock, TX (US)

(21) Appl. No.: 14/684,022

(22) Filed: Apr. 10, 2015

(51) Int. Cl. G01N 33/94; G01N 33/14; G01N 21/78

(52) U.S. Cl. CPC G01N 33/94; G01N 21/78; G01N 33/14; G01N 2021/7796

(57) ABSTRACT

Embodiments of portable chemical testing apparatus, methods, and systems may provide information indicative of the presence of at least one target chemical in a liquid to be tested, such as a drink, and may include a portable housing having a storage compartment for containing dispensable personal care material and a chemical testing platform which may be brought into contact with liquid to be tested.

### CROSS-REFERENCE TO RELATED APPLICATIONS

N/A

### FIELD OF THE INVENTION

The disclosure relates to portable chemical detection apparatus and methods for use in public places.

### BACKGROUND OF THE INVENTION

This disclosure relates to apparatus, systems, and methods for the detection of chemicals and other substances such as drugs or narcotics, as well as other chemicals used in clandestine activities.

Conventional systems tend to be large and immobile. In addition, such systems can require users to manually apply toxic chemicals as testing agents. As a result, these systems are not mobile, are hard to use, and are not appropriate for use in testing beverages for personal consumption. Hence, adoption of conventional systems for field use has been limited at best.

Various techniques for detecting potentially harmful chemicals such as drugs or other narcotics have been developed. These range from sniffing dogs to highly sophisticated vapor detection devices. Machine detection of substances has primarily focused on the detection of explosive substances, particularly with military applications in mind. Detection of such substances can be accomplished through methods such as x-ray or gamma ray detection and magnetic resonance imaging. Vapor based detection methods include electron capture, gas chromatography, bio-sensor, and laser photo-acoustic detection. These methods of detection, however, are more suitable for detection in a way that is not discreet and using a system or apparatus that is not easily portable.

Other chemical testing apparatus are known for use in laboratory, clinical, and industrial environments. As with the systems and methods described above, these apparatus are not intended to be discreet. Rather, these systems tend to be large and immobile.

### BRIEF SUMMARY OF THE INVENTION

The portable chemical testing apparatus may be configured for selectable exposure to a drink or drink mixture in order to provide information related to whether one or more target chemicals is present in the drink or drink mixture. The portable chemical testing apparatus may include a portable housing. The portable housing includes a storage compartment having a nominal volume of, for example, less than fifty milliliters. Various volumes and arrangements of the storage compartment are possible depending on factors such as the intended type of chemical testing. The storage compartment may be configured to hold or distribute one or more personal care materials such as cosmetics, lip balm, lip gloss, lip stick, mascara, eye liner, perfume, cologne, lotion, hand sanitizer, or breath spray.

The storage compartment may also be configured with a removable or replaceable cap for opening and closing the storage compartment, retaining personal care material, or dispensing personal care material. A chemical testing platform can be supported by the portable housing or the cap. In some embodiments, the chemical testing platform can be supported by the portable housing or the cap in movable relation relative to the chemical testing apparatus. In addition, the chemical testing platform can be supported by either the portable housing or the cap in a fixed relationship thereto.

The chemical testing platform can be fixed or movable relative to the portable housing and the cap. In some embodiments, chemical testing platform can be fixed or adhered to the portable housing or the cap of the chemical testing platform. In such an embodiment, the chemical testing platform may be fixed or adhered to at least one surface of the portable housing or the cap. In some embodiments, the chemical testing platform can be configured to collect or retain a sample upon coming into contact with the drink or drink mixture.

Alternatively, in embodiments in which the chemical testing platform is movable relative to the portable housing and the cap, the chemical testing platform can be selectively moved between a stored position and an exposed position. In such an embodiment, the chemical testing platform can be configured for contacting the drink or drink mixture in the exposed position. In some embodiments, the chemical testing platform can be configured to collect or retain a sample upon coming into contact with the drink or drink mixture.

In some embodiments, the chemical testing platform can be removable relative to the portable housing or the cap between a stored position and an exposed position. In such an embodiment, the chemical testing platform can be configured for contacting the drink or drink mixture in the exposed position. In some embodiments, the chemical testing platform can be configured to collect or retain a sample upon coming into contact with the drink or drink mixture.

The portable chemical testing apparatus can include a portable applicator. The portable applicator may be configured for manual manipulation relative to the storage area in order to remove one or more personal care materials from the storage area. For example, the portable applicator may be of the wand or brush type and be configured to dispense lip gloss. The portable applicator can also be a portable applicator analog. The portable applicator analog may be configured to support the chemical testing platform by, for example, mimicking the portable applicator.

The portable chemical testing apparatus may be configured to have a second storage compartment. In such an embodiment, the chemical testing apparatus includes a housing, a 100 cap, or both. The second storage compartment can be configured to hold the chemical testing platform. Alternatively, the portable chemical testing apparatus may be configured to include a second housing, a second cap, or both. In such an embodiment, the second housing or second cap includes a second storage 105 compartment that may be configured to hold the chemical testing platform. The portable chemical testing apparatus of this type can be assembled to provide a housing assembly. The housing assembly can include the first storage compartment and the second storage compartment.

The chemical testing platform can be configured in a number of ways depending on, for example, the shape and size of the portable chemical testing apparatus or the type of chemical test desired. The chemical testing platform can include one or more of the following: an assay, an assay panel, an immunoassay panel, a competitive immunoassay panel, a competitive immunoassay panel dip device, a competitive immunoassay panel test strip, a qualitative detector of at least one chemical metabolite, a chemical test media, a circuit responsive to at least one chemical metabolite, and a micro-assay integrated circuit.

### BRIEF DESCRIPTION OF THE DRAWINGS

The novel features believed characteristic of the disclosed subject matter will be set forth in any claims that are filed. The disclosed subject matter itself, however, as well as a preferred mode of use, further objectives, and advantages thereof, will best be understood by reference to the following detailed description of an illustrative embodiment when read in conjunction with the accompanying drawings, wherein:

FIG. 1 is a front perspective view of portable chemical testing apparatus according to an embodiment.

FIG. 2 is a front perspective view of portable chemical testing apparatus with the chemical testing platform removed from the portable housing and cap.

FIG. 3 is a perspective view of test media suitable for use in embodiments.

FIG. 4 is an exemplary illustration of the chemical testing platform being exposed to a drink or drink mixture.

FIG. 5 is an example of a movable chemical testing platform in the stored position.

FIG. 6 is an example of a movable chemical testing platform in the exposed position.

FIG. 7 is an example of an alternative embodiment of a movable chemical testing platform in the stored position.

FIG. 8 is an example of an alternative embodiment of a movable chemical testing platform in the exposed position.

FIG. 9 is an embodiment of the portable chemical testing apparatus including a removable cap attached to the apparatus to conceal the chemical testing platform.

FIG. 10 illustrates the apparatus of FIG. 9 with the cap removed from the portable chemical testing apparatus such that the chemical testing platform is not concealed.

FIG. 11 is an embodiment of the portable chemical testing apparatus including an alternative, attached cap used to conceal the chemical testing platform.

FIG. 12 illustrates the apparatus of FIG. 11 with the cap in the open position such that the chemical testing platform is not concealed.

FIG. 13 is an embodiment of the portable chemical testing apparatus including a removable cap attached to the apparatus in which the chemical testing platform is not attached to or supported by the cap or the portable housing.

FIG. 14 illustrates the apparatus of FIG. 13 with the cap removed from the portable chemical testing apparatus such that the chemical testing platform is not concealed.

FIG. 15 is an embodiment of the portable chemical testing apparatus including an alternative removable cap in which the chemical testing platform is not attached to or supported by the cap or the portable housing.

FIG. 16 illustrates the apparatus of FIG. 15 with the cap attached to the portable housing.

FIG. 17 is an embodiment of the portable chemical testing apparatus including a rotating cap in which the chemical testing platform is not attached to or supported by the cap or the portable housing.

FIG. 18 illustrates the apparatus of FIG. 17 with the cap rotated into a closed position such that the contents of the storage compartment are prevented from being dispensed.

FIG. 19 illustrates portable chemical testing apparatus according to an embodiment.

FIG. 20 is a partial sectional view of portable chemical testing apparatus shown generally in FIG. 19.

### DETAILED DESCRIPTION OF ILLUSTRATIVE EMBODIMENTS

The present disclosure includes embodiments of apparatus, systems and methods of testing for chemical substances. In embodiments, for example, subject chemical substances may be substances commonly known as date rape drugs for rendering a target individual helpless, unconscious, without memory, or incapable of distinguishing adverse circumstances, intoxicated, or in a state involving less than ordinary self-control. As used herein, “testing” includes, without limitation, the providing, reacting, storing, displaying, obtaining, profiling, measuring, detecting, sensing, completing, indicating or performing of any test, protocol, reaction, media, profile, sensing, sensor, indicator, indication, detector, detection, result, or measurement of the presence, absence, concentration, activity, reactivity, availability, reaction, reaction mechanism, property, state, change, stability, appearance, disappearance, function, effect, effectiveness, activity, reactivity, reaction, or reaction mechanism for any chemical, compound, mixture, structure, molecule, element, atom, agent, functional group, bond, form, substance or material, whether synthesized, occurring in nature, concentrated or extracted from any organic or inorganic material, source, micro-organism, animal or plant. It will be understood by one of ordinary skill that embodiments disclosed herein may provide apparatus, systems and methods for testing for the presence of substances commonly known as date rape drugs including for example, but not limited to the following: benzodiazepines (including Rohypnol, Valium and Xanax), GHB (gamma-hydroxyl-butyrate), and ketamine. It will be understood that other substances can be tested.

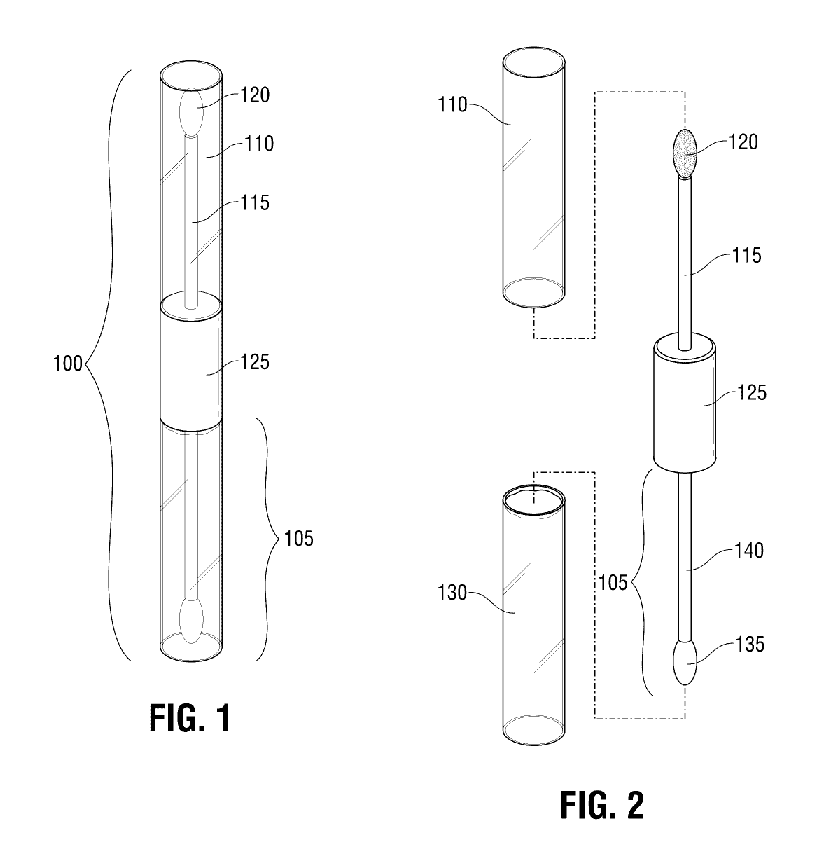

Illustrated in FIG. 1 is an apparatus 100 for chemical testing according to an embodiment. It will be understood that apparatus 100 may be of different arrangement or appearance, may have different dimensions, or may be formed of different materials. Apparatus 100 may include a portable housing 110 including a storage compartment 115. The storage compartment 115 may have a nominal volume less than substantially fifty milliliters. Storage compartment 115 may be configured to hold dispensable personal care material. One of ordinary skill will understand that personal care material can include any personal care material to be dispensed to a user. For example, personal care material may include, without limitation, lip stick, lip gloss, mascara, or eye liner. FIG. 1 illustrates a portable chemical testing apparatus 100 including a lip gloss wand 145 and lip gloss applicator 120 for application of personal care material that is lip gloss.

One of ordinary skill will understand that, in various embodiments, the nominal volume of the first storage compartment 115 can be substantially identical to the nominal volume of a second storage compartment 140 of a second portable housing 130 configured for dispensing personal care material that is identical to the personal care material dispensable from the first storage compartment 115. In other embodiments including a first and second portable housing, the personal care materials may not be identical.

A cap 125 is positioned between a first storage compartment 115 and a second storage compartment 140. In the embodiment illustrated by FIG. 1, the personal care material, lip gloss wand 145 and lip gloss applicator 120 are supported by the first cap 125.

FIG. 2 illustrates apparatus 100 for chemical testing with portable housing 110 and second portable housing 130 removed from contact with the cap portion 125. In this embodiment, the personal care material (lip gloss wand 145 and lip gloss applicator 120) is exposed and ready for use. In addition, a chemical testing platform 105 is exposed and ready for use. In the illustrated embodiment, chemical testing platform 105 includes a portable housing 130 for selectable exposure of the chemical testing platform 105, a faux personal care material wand 150, and a chemical testing platform portion 135 configured for selectable exposure to a drink mixture.

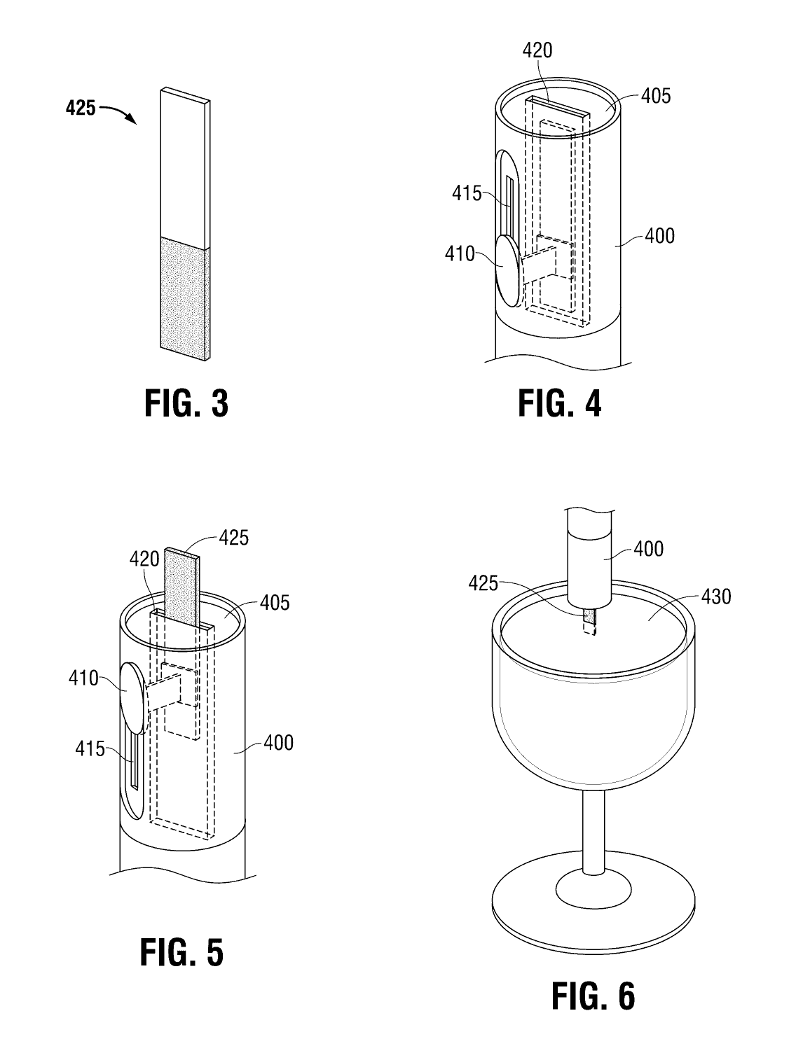

FIG. 3 is a perspective view of test media 425 suitable for use in embodiments. In the particular embodiment shown in FIG. 3, the test media 425 includes an elongated chemical test substrate or strip adapted for contacting a liquid to be tested for at least one substance. It will be understood that, in embodiments (not shown), the test media 425 may be configured for testing for multiple substances.

FIG. 4 illustrates a portion of an embodiment of the apparatus for chemical testing 100. The apparatus for chemical testing 100 includes a cap 400 with a cap top portion 405 and a slot 420. A chemical testing platform 425 can be selectably movable between a stored position and an exposed position. The position of the chemical testing platform 425 can be selected using position selection arm 410. Position selection arm 410 slides along selection arm guide track 415. The position of the position selection arm 410 is such that the chemical testing platform 425 is in the stored position in this illustration.

FIG. 5 also illustrates a portion of an embodiment of the apparatus for chemical testing 100. In this illustration, the position selection arm 410 is moved along the selection arm guide track 415 such that the chemical testing platform 425 has moved through the slot 420 in the cap top portion 405 and is in the exposed position.

FIG. 6 illustrates the embodiment of FIG. 5, including cap 400 and chemical testing platform 425 in the exposed position. The chemical testing platform 425 is being exposed to a drink mixture 430. In this illustration, chemical testing platform 425 is configured for selectable exposure to the drink mixture 425 to provide information indicative of the presence of at least one target chemical in the drink mixture 425.

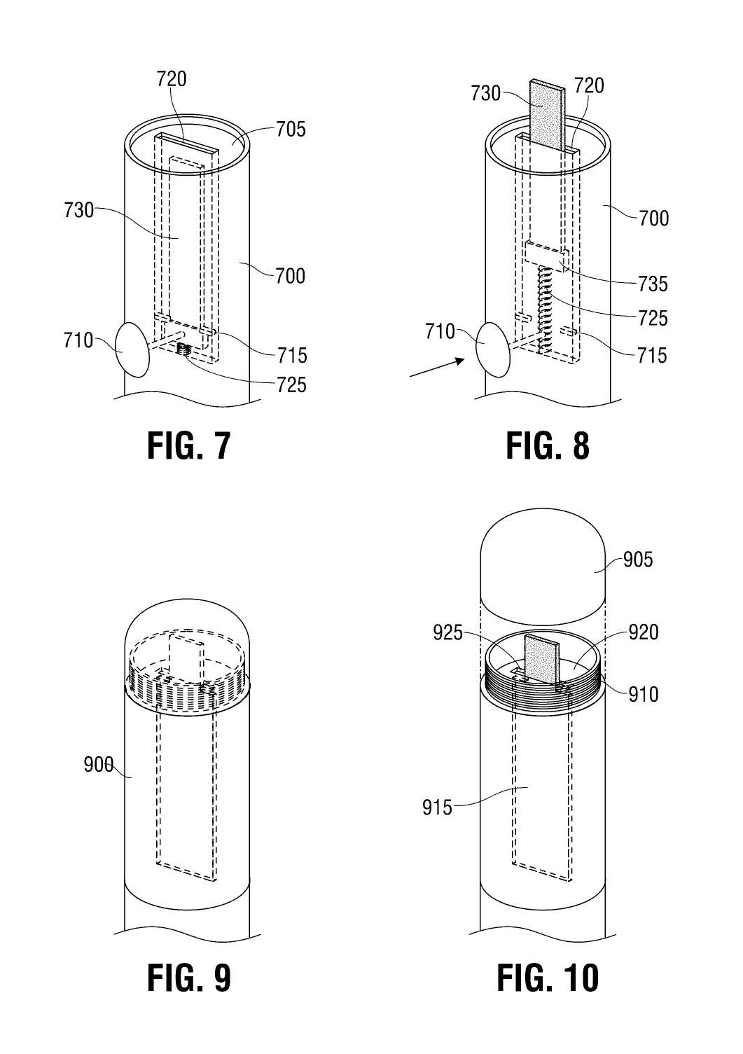

FIG. 7 illustrates a portion of an embodiment of the apparatus for chemical testing 100. The apparatus for chemical testing 100 includes a cap 700 with a cap top portion 705 and a slot 720. A chemical testing platform 730 can be selectably movable between a stored position and an exposed position. The position of the chemical testing platform 730 can be selected using position selection lever 710. Position selection lever 710 is in mechanical communication with a spring 725. The spring 725, as illustrated here, is compressed such that the chemical testing platform 720 is in the stored position. A stopper 735 is attached to the bottom portion of the chemical testing platform 720. The stopper 735 rests on the travel containment arm 715 when the chemical testing platform 730 is in the stored position.

Position selection lever 710 can be in an “in” or “out” position corresponding to an exposed or stored position, respectively. In this illustration, position selection lever 710 is in the “out” position such that the chemical testing platform 730 is in the stored position. It should be understood that in alternative embodiments, the position of the position selection lever 710 and the position of the chemical testing platform 730 may not correspond to those illustrated here.

FIG. 8 also illustrates a portion of an embodiment of the apparatus for chemical testing 100. In this illustration, the position selection lever 710 is in the “in” position such that the spring 725 has expanded and the chemical testing platform 730 has moved through the slot 720 in the cap top portion 705 and is in the exposed position.

FIGS. 9 and 10 illustrate a portion of an embodiment of portable apparatus for chemical testing 900. The chemical testing platform 915 is supported by the cap 900. The cap 900 has a cap top portion 920 and a slot 925. The chemical testing platform 915 is positioned such that a portion of the chemical testing platform 915 is contained within the cap 900, while another portion of the chemical testing platform 915 is positioned above the cap top portion 920.

A second cap 905 can be attached to the cap 900 via an attachment means 910. Attachment means 910 can be any known means of attachment such as, for example, a threaded, screw-type attachment as illustrated. When the second cap 905 is attached to the cap 900 via the attachment means 910, as illustrated in FIG. 9, the chemical testing platform 915 is enclosed within the cap 900 and the second cap 905 and is not exposed. When the second cap 905 is removed from cap 900, as illustrated in FIG. 10, a portion of the chemical testing platform 915 is exposed and can be selectably exposed to a drink mixture to provide information indicative of the presence of at least one target chemical in the drink mixture.

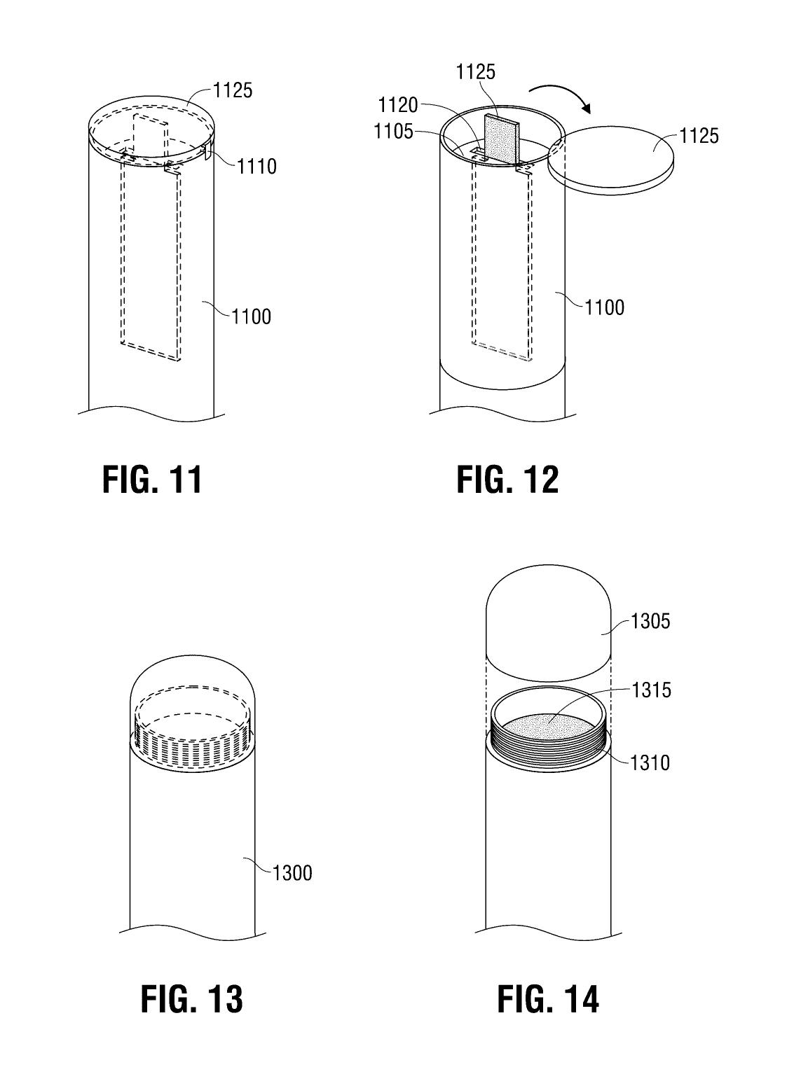

FIGS. 11 and 12 illustrate a portion of an embodiment of the apparatus for chemical testing 100. The chemical testing platform 1115 is supported by the cap 1100. The cap 1100 has a cap top portion 1105 and a slot 1120. The chemical testing platform 1115 is positioned such that a portion of the chemical testing platform 1115 is contained within the cap 1100, while another portion of the chemical testing platform 1115 is positioned above the cap top portion 1105. In this embodiment, the walls of the cap 1100 extend above the cap top portion 1105.

A second cap 1125 is attached to the cap 1100 via a rotating attachment means 1110. Rotating attachment means 1110 can be any known means of rotating attachment such as, for example, a hinge as illustrated. When the second cap 1125 is attached to the cap 1100 via the rotating attachment means 1125 and in a closed position as illustrated in FIG. 11, the chemical testing platform 1115 is enclosed within the cap 1100 and the second cap 1125 and is not exposed. When the second cap 1125 is in an open position as illustrated in FIG. 12, a portion of the chemical testing platform 915 is exposed and can be selectably exposed to a drink mixture to provide information indicative of the presence of at least one target chemical in the drink mixture.

FIGS. 13 and 14 illustrate a portion of an embodiment of the apparatus for chemical testing 100. In this embodiment, the cap 1300 includes a substantially hollow portion configured for retaining a sample of a drink mixture upon contacting the same. A second cap 1305 can be attached to the cap 1300 via an attachment means 1310. Attachment means 1310 can be any known means of attachment such as, for example, a threaded, screw-type attachment as illustrated.

When the second cap 1305 is removed from the cap 1300, as illustrated in FIG. 14, a porous retention means 1315 is exposed. The porous retention means 1315 is supported by the cap 1300 and is configured to retain a sample of the drink mixture in the substantially hollow portion of the cap 1300 or in the porous retention means 1315 itself upon contact with the drink mixture.

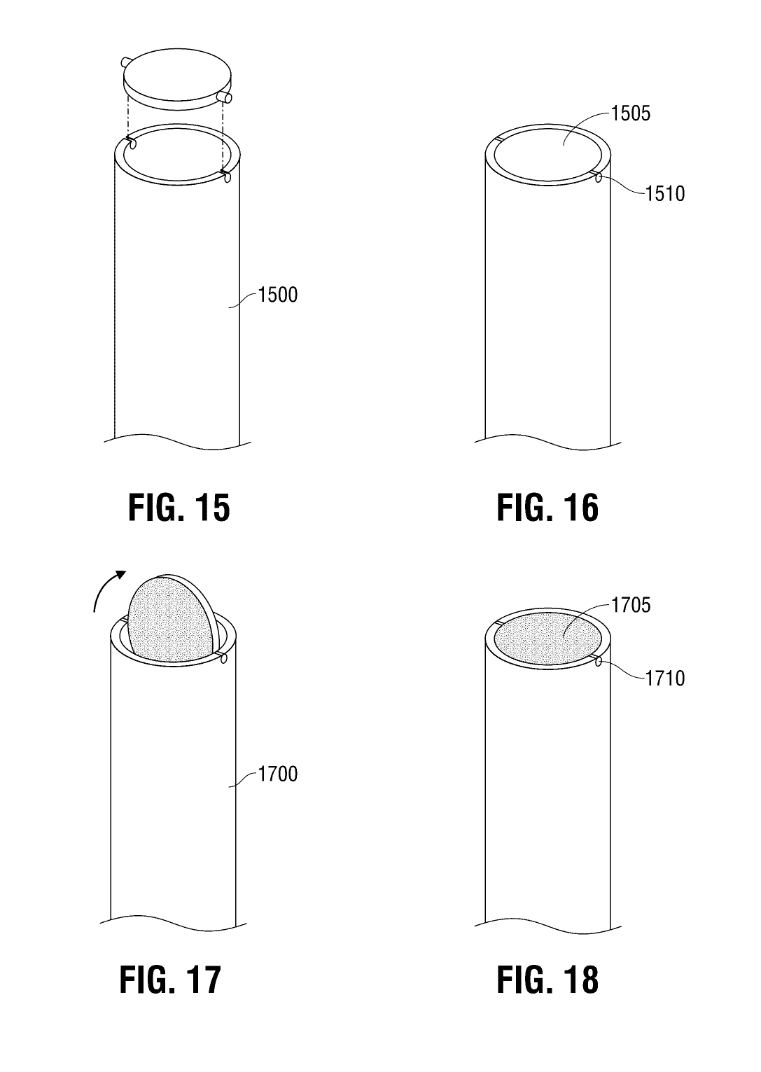

FIGS. 15 and 16 illustrate a portion of an embodiment of the apparatus for chemical testing 100. In this embodiment, the cap 1500 includes a substantially hollow portion configured for retaining a sample of a drink mixture upon contacting the drink mixture. A retention means 1505 can be attached to the cap 1500 via an attachment means 1510. Attachment means 1510 can be any known means of attachment such as, for example, the clip-in type attachment as illustrated. FIG. 15 depicts the retention means 1505 removed from contact with the cap 1500. FIG. 16 depicts the retention means 1505 attached to the cap 1500 via the attachment means 1510.

FIGS. 17 and 18 illustrate a portion of an embodiment of the apparatus for chemical testing 100. In this embodiment, the cap 1700 includes a substantially hollow portion configured for retaining a sample of a drink mixture upon contacting the drink mixture. A retention means 1705 can be rotatably attached to the cap 1700 via a rotating attachment means 1710. The rotating attachment means 1710 can be any known means of rotating attachment such as, for example, a hinge. FIG. 17 depicts the retention means 1705 partially rotated such that the substantially hollow portion of the cap 1700 is exposed and can collect and retain a sample of a drink mixture. FIG. 18 depicts the retention means 1705 in a closed position. It should be understood that the retention means 1705 could be made of a porous or a non-porous material such that a drink mixture may or may not be collected and retained upon contacting the drink mixture while the retention means is in a closed position as illustrated in FIG. 18.

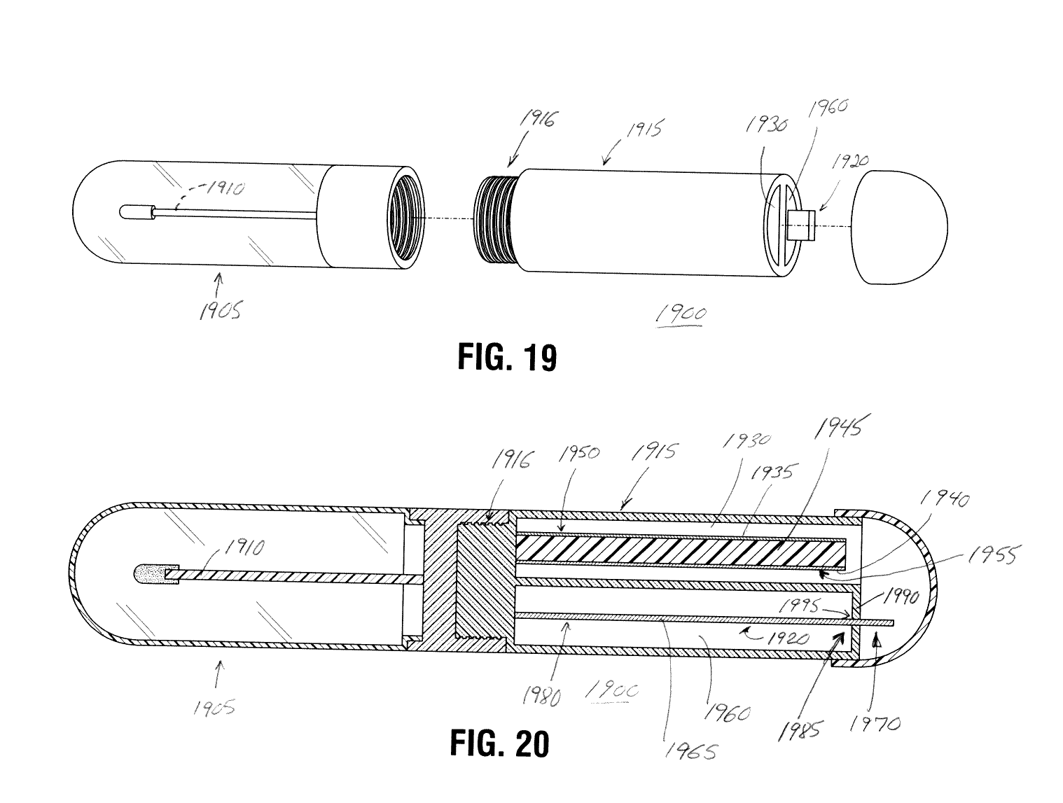

FIG. 19 illustrates portable chemical testing apparatus 1900 according to an embodiment. Portable chemical testing apparatus 1900 shown in FIG. 19 may be substantially identical to portable apparatus for chemical testing 900 shown in FIGS. 9-10, except as otherwise described herein or illustrated in FIGS. 19-20. FIG. 20 is a partial sectional view of portable chemical testing apparatus shown generally in FIG. 19. As shown in FIG. 19, in an embodiment portable chemical testing apparatus 1900 may include a first storage compartment 1905 for containing personal care material such as lip gloss and housing an applicator 1910 such as a lip gloss applicator. As shown in FIG. 19, in an embodiment portable chemical testing apparatus 1900 may include a portable chemical testing platform housing 1915 supporting multiple chemical testing platforms 1920 in respective chemical testing platform compartments 1925. As shown in FIG. 19, portable chemical testing platform housing 1915 may be removably fixed to first storage compartment 1905, such as by a threaded connection 1916, to provide access to compartments of portable chemical testing platform housing 1915 for installing or removing chemical testing platforms in such compartments. As best shown in FIG. 20, in the particular embodiment illustrated in FIGS. 19-20, portable chemical testing platform housing 1915 includes a first chemical testing platform compartment 1930 supporting and housing first chemical testing platform 1935 and second chemical testing platform 1940. First chemical testing platform compartment 1930 may be open at the end to permit a liquid to be tested, such as a beverage, to be introduced or flow into the first chemical testing platform compartment 1930 such as when dipped into such liquid to be tested. It will be understood that liquid to be tested flowing into the chemical testing platform compartment 1930 may contact both first chemical testing platform 1935 and second chemical testing platform 1940, such that the same are partially or wholly immersed in such liquid to be tested for a desired exposure period. It will be understood that first chemical testing platform 1935 and second chemical testing platform 1940 may be supported by being affixed, for example, to opposite surfaces of a supporting fixture 1945 such as, for example, a plastic stick. In a particular embodiment as shown in FIG. 20., first chemical testing platform 1935 may include, for example, a ketamine test strip 1950 affixed to a respective surface of the supporting fixture 1945 by compatible adhesive material that does not interact with the test strip so as to provide a false result. It will be understood that, in an embodiment, suitable adhesive material for affixing ketamine test strip 1950 to supporting fixture 1945 may be a non-amine containing adhesive material.

In a particular embodiment as shown in FIG. 20., second chemical testing platform 1940 may include, for example, a gamma-hydroxybutyric acid (GHB) test strip 1955 affixed to a respective surface of the supporting fixture 1945 by compatible adhesive material that does not interact with the test strip so as to give a false result.

Referring to FIG. 20, in the particular embodiment illustrated, portable chemical testing platform housing 1915 includes a second chemical testing platform compartment 1960 supporting and housing third chemical testing platform 1965. Second chemical testing platform compartment 1960 may be closed to prevent liquid to be tested, such as a beverage, from flowing into the second chemical testing platform compartment 1960 when dipped into such liquid to be tested. It will be understood that liquid to be tested without flowing into the second chemical testing platform compartment 1960 may contact third chemical testing platform 1965 only at an exposed absorbent portion 1970 of the third chemical testing platform 1965, such that liquid to be tested absorbed into the exposed absorbent portion 1970 enters into the third chemical testing platform 1965 by capillary action through media of the third chemical testing platform 1965, such that a portion of the third chemical testing platform 1965 remains isolated in the second chemical testing platform compartment 1960 without contacting liquid at a surface portion 1980 thereof. It will be understood that surface portion 1980 thus isolated in second chemical testing platform compartment 1960 contacts only air contained in the second chemical testing platform compartment 1960, and thus is not immersed in liquid to be tested. In a particular embodiment as shown in FIG. 20., third chemical testing platform 1965 may include, for example, a benzodiazepine (BZO) test media device. In an embodiment, a suitable BZO test media device may include a BZO immunoassay test device. It will be understood that a seal 1985 may be formed by engagement between third chemical testing platform 1965 and an end wall 1990 having defined therein an opening or slot 1995, through which exposed absorbent portion 1970 extends outside of the second chemical testing platform compartment 1960 for absorbing liquid to be tested while also preventing the liquid from flowing into the second chemical testing platform compartment 1960 when dipped into such liquid to be tested. It will be understood that in an embodiment (not shown), media or material of third chemical testing platform 1965 may define a shoulder around slot 1995 and abutting end wall 1990 to form such an enlarged seal 1985 to prevent liquid from flowing into second chemical testing platform compartment 1960.

1. Apparatus for chemical testing, said apparatus comprising:
a chemical testing platform configured for selectable exposure to a drink mixture to provide information indicative of the presence of at least one target chemical in the drink mixture;
a portable housing comprising a first storage compartment, the first storage compartment having a nominal volume less than 50 milliliters, the first storage compartment configured to hold dispensable personal care material;
a first cap selectively positionable relative to the first storage compartment between a closed position and an open position, in the closed position the first cap cooperating with the portable housing to prevent dispensing of the dispensable personal care material from the first storage compartment;
the chemical testing platform supported by at least one of the portable housing and the first cap.

2. Apparatus for chemical testing according to claim 1 and further comprising:
the chemical testing platform supported by said at least one of the portable housing and the first cap in movable relationship relative thereto.

3. Apparatus for chemical testing according to claim 2 and further comprising:
the chemical testing platform being selectably movable relative to said at least one of the portable housing and the first cap between a stored position and an exposed position, the chemical testing platform configured for contacting the drink mixture in the exposed position.

4. Apparatus for chemical testing according to claim 3 and further comprising:
the chemical testing platform configured for retaining a sample of the drink mixture upon contacting the same.

5. Apparatus for chemical testing according to claim 1 and further comprising:
the chemical testing platform supported by said at least one of the portable housing and the first cap in removable relationship relative thereto.

6. Apparatus for chemical testing according to claim 5 and further comprising:
the chemical testing platform being selectably removable relative to said at least one of the portable housing and the first cap between a stored position and an exposed position, the chemical testing platform configured for contacting the drink mixture in the exposed position.

7. Apparatus for chemical testing according to claim 6 and further comprising:
the chemical testing platform configured for retaining a sample of the drink mixture upon contacting the same.

8. Apparatus for chemical testing according to claim 1 and further comprising:
the chemical testing platform supported by said at least one of the portable housing and the first cap in fixed relationship relative thereto.

9. Apparatus for chemical testing according to claim 1 and further comprising:
a portable applicator configured for manual manipulation relative to the first storage area to remove an application of personal care material from the first storage area.

10. Apparatus for chemical testing according to claim 9 and further comprising:
a portable applicator analog configured to support the chemical testing platform, the portable applicator analog configured to mimic the portable applicator.

11. Apparatus for chemical testing according to claim 1 and further comprising:
the chemical testing platform adhered to at least one surface of the at least one of the portable housing and the first cap.

12. Apparatus for chemical testing according to claim 1 and further comprising:
the chemical testing platform comprising at least one of the following:
an assay, an assay panel, an immunoassay panel, a competitive immunoassay panel, a competitive immunoassay panel dip device, a competitive immunoassay panel test strip, a qualitative detector of at least one chemical metabolite, a chemical test media, a circuit responsive to at least one chemical metabolite, a micro-assay integrated circuit.

13. Apparatus for chemical testing according to claim 1 and further comprising:
the personal care material comprising the following:
cosmetics, lip balm, lip gloss, lip stick, mascara, eye liner, perfume, cologne, lotion, hand sanitizer, breath spray.

14. Apparatus for chemical testing according to claim 1 and further comprising:
the at least one of the housing and the first cap comprising a second storage compartment configured to hold the chemical testing platform.

15. Apparatus for chemical testing according to claim 1 and further comprising:
at least one of a second housing and a second cap comprising a second storage compartment configured to hold the chemical testing platform;
the at least one of the housing and the first cap configured to be assembled together with the at least one of a second housing and a second cap to provide a housing assembly, the housing assembly comprising the first storage compartment and the second storage compartment.

16. A system for chemical testing, said system comprising:
a chemical testing platform configured for selectable exposure to a drink mixture to provide information indicative of the presence of at least one target chemical in the drink mixture;
a portable housing defining a first storage compartment, the first storage compartment having a nominal volume less than 50 milliliters, the first storage compartment configured to hold dispensable personal care material;
a first cap selectively positionable relative to the first storage compartment between a closed position and an open position, in the closed position the first cap cooperating with the portable housing to prevent dispensing of the dispensable personal care material from the first storage compartment;
the at least one of the portable housing and the first cap configured to support the chemical testing platform.

17. A method for chemical testing, said system comprising:
providing a chemical testing platform configured for selectable exposure to a drink mixture to provide information indicative of the presence of at least one target chemical in the drink mixture;
providing a portable housing defining a first storage compartment, the first storage compartment having a nominal volume less than 50 milliliters, the first storage compartment configured to hold dispensable personal care material; and
providing a first cap selectively positionable relative to the first storage compartment between a closed position and an open position, in the closed position the first cap cooperating with the portable housing to prevent dispensing of the dispensable personal care material from the first storage compartment, at least one of the portable housing and the first cap configured to support the chemical testing platform.
